(19) United States
(12) Patent Application Publication
(10) Pub. No.: US 2021/0239746 A1
(43) Pub. Date: Aug. 5, 2021

(54) SYSTEM AND METHOD FOR IDENTIFYING THE CONDITION OF FLUIDS INSIDE A CONTAINER

(71) Applicant: Robert Bosch Limitada, Campinas - SP (BR)

(72) Inventors: Fernando Lepsch, Campinas - SP (BR); Marcello Francisco Brunocilla, Indaiatuba - SP (BR)

(21) Appl. No.: 17/130,882

(22) Filed: Dec. 22, 2020

(30) Foreign Application Priority Data
Dec. 26, 2019 (BR) 102019027850-1

(51) Int. Cl. G01R 27/22; F02D 41/00; F02D 41/14

(52) U.S. Cl. CPC G01R 27/22; F02D 41/1496; F02D 41/1494; F02D 41/0025

(57) ABSTRACT

A system and method for identifying the condition of fluids inside a container, by means of a resistive element associated with a control unit of the resistive element, preferably used for identifying the condition of water for application in a fuel vehicle water injection system, but not limited to this application. The control unit of the resistive element is comprised of a processing unit capable of obtaining the reading of the resistance of a resistive element and processing the information for performing the identification of the condition of the fluid, and also the composition can use the resistive element as a heating element through a heater control unit when it is identified that the condition of the fluid is solid.

### BACKGROUND

The present invention refers to a system and method for identifying the condition of fluids inside a container, by means of a resistive element associated with a control unit of the resistive element, preferably used to identify the condition of water for application in a water injection system in combustion vehicles, but not limited to this application.

In addition to identifying the condition of fluids, the present invention allows the use of the resistive element as a heating element when the condition of the fluid is solid.

In order to mitigate greenhouse gas emissions and to reduce dependency on fossil fuels, several alternative technologies have been developed and made available for vehicles with internal combustion engines. However, the best solution should take into account the geographical and socioeconomic characteristics of each country, as well as its energy matrix, its carbon emissions legislation, and the environmental impact of fuel throughout its life cycle.

Brazil has a strong reputation for its fleet of bi-fuel vehicles, a long-time experience in use of ethanol as fuel and a broad distribution network. This sets it apart from other global markets and justifies a unique approach to reducing CO₂ emissions.

However, there are some limitations in the use of bi-fuel engines, popularly known as “flex” engines. To meet the demand for the use of two fuels in a single tank, the regulation of a “flex” engine tends to be intermediate, since the regulation of single-fuel engines is different, the fuel being ethanol or gasoline. This is because the vast majority of bi-fuel engines usually have a single geometric compression ratio, since this ratio is directly linked to the upward and downward strokes of the pistons.

In its course, the piston reaches a higher point and a lower point in its stroke, respectively called top dead center (TDC) and bottom dead center (BDC). In this way, the operation of the engine of a passenger vehicle has four strokes: intake, compression, combustion and exhaust.

The compression rate occurs during the second stroke: the intake valves close after the injection of the air/fuel mixture, and the latter is compressed for combustion and exhaust to occur. Accordingly, the engine compression rate is obtained, which is the ratio between the volume of the combustion chamber of the piston in its bottom dead center BDC (higher volume) and its top dead center TDC (lower volume).

Gasoline engines often use lower ratios (typically between 8:1 and 12:1), while ethanol-powered engines work best at higher ratios (12:1 or even 14:1).

On the other hand, bi-fuel engines operate at an intermediate ratio and may vary according to the manufacturers of thrusters. Those who prioritize performance with gasoline, offering the option with ethanol only for market reasons, opt for lower rates, between 10:1 and 11.5:1. This fact can be noticed when observing the power and torque figures with both fuels, where for example a propellant that delivers 144 horsepower with ethanol and 141 with gasoline. However, when one observes consumption, the figures are very high with alcohol (5.5 km/l) and considered good with gasoline (9 km/l). In this way, the performance with ethanol is impaired to the detriment of gasoline, wherein using ethanol becomes advantageous only with a large drop in its price, to values 65% less than the price of gasoline, a condition that does not occur often. Therefore, the driver tends to fill up his vehicle usually with gasoline.

On the other hand, engines designed to work with ethanol use higher compression ratios, higher than 13:1. As alcohol has greater knocking resistance, it accepts greater compression without loss of performance. However, the drop in performance appears when the engine runs on gasoline, which has its calibration with reduced torque and power figures to avoid knocking, extremely detrimental to the durability of the engine. There are large variations in power and torque figures in engines designed to run on ethanol, in an illustrative example, 111 horsepower for ethanol operation and 104 horsepower when run on gasoline. Thus, it is feasible when the price of ethanol is 75% or up to 80% of the oil derivative, as the consumption figures are very close with both fuels, such as 7.5 km/l for ethanol and 9.5 for gasoline.

Thus, an improvement in the use of bi-fuel engines with advantages in fuel economy (when any fuel is used), increased performance and consequent reduction of CO₂ emissions is achieved by a bi-fuel engine (originally designed to operate with ethanol and is propelled only with gasoline or any fuel mixture) combining high compression rate technology with the injection of a coolant.

It should be noted that the injection of coolant into internal combustion engines is an effective means of changing or reducing the knocking limits (to avoid knocking) of bi-fuel engines when running on gasoline only. The use of coolant injection allows an internal combustion propellant to be optimized in its operation with ethanol, without loss of efficiency when propelled with gasoline as well. Coolant can also be used in the engine when propelled with gasoline, when subjected to knocking phenomena in more severe conditions (supercharged engines, high compression ratios, racing engines etc.), and also for the protection of its components.

In order to be able to balance the bi-fuel engines upon propelling, both with ethanol and gasoline and thus extracting more power and torque from the engine with lower fuel consumption, reducing pollutant emissions in its normal operation (by increasing the consumption ratio between ethanol and gasoline above 69%, potentially up to 80%), a coolant (e.g. water) is injected into the engine during its operation.

In order to efficiently achieve the increase in power extracted from the engine associated with lower gasoline consumption and consequent reduction of emitted CO₂, the water to be injected must be free of contaminants, mineral salts (demineralized) and electrical loads (deionized). Such a condition is imperative for the preservation of the internal components of the system against corrosion, obstructions, and clogging.

However, under current conditions, the use of water commonly found in homes, water tanks, and taps is not feasible for this type of application, since it contains microcontaminants, solid impurities, and mineral salts.

Microcontaminants, solid impurities, and mineral salts found in water can cause serious damage to the coolant injection system. The high electrical conductivity of mineral salts can cause corrosion, wear in components, and sediment deposition. Metal particles (“rust”) from the oxidation of residential water ducts, as well as the presence of sand, earth or other inorganic elements (also coming from the water pipe) can lead to the blockage of mechanical parts of the water injection system. In addition, the presence of biological agents can clog water lines and cause microbiological corrosion attack on plastics and metals.

For this application condition to be achieved, the water must be pure, filtered, and deionized. However, it is known that this type of water is not easily found in emerging markets and, when found, its price is quite high. It is therefore necessary to ensure that the water to be used is not unfit for this type of application.

The patent document CN201594084 reveals a circuit for detection of water quality using the reading of its resistance, by means of two electrodes at a fixed distance. Despite presenting a simple and low cost solution for application in detection of drinking water, it is not suitable for use in coolant injection systems in vehicles, since it is necessary to monitor other characteristics of the fluid, such as, for example, whether it is in solid state due to low temperatures.

It is important to know under which conditions the water is considered to be in a clean state. The desirably observed conditions may comprise the current physical state of the clean water, whether some type of treatment or filtration will be required in order for it to actually meet the conditions of cleanliness, demineralization, and/or deionization necessary so as not to compromise the water injection system.

Thus, in order to mitigate the technical limitations of the use of previous solutions and ensure better safety in the use of fluids in water injection systems, the present invention arises.

### SUMMARY

Thus, the main objective of the present invention is to describe a system and method for identifying the condition of fluids inside a container, by means of a resistive element associated with a control unit of the resistive element, preferably used to identify the condition of water for application in a water injection system in combustion vehicles, but not limited to this application.

Additionally, it is an objective of the present invention to provide a system and method that performs the identification of fluid condition by reading the voltage in a resistive element associated with the fluid.

Furthermore, another objective of the present invention is to disclose a system and method that, upon identifying the solid condition of the fluid, caused for example by low temperature, triggers the resistive element associated with the fluid to thus increase the temperature and making it usable for the injection system.

Moreover, it is an objective of the present invention to disclose a system and method capable of identifying the condition of the fluid through the association with a lower limit and an upper limit of resistivity of the resistive element.

All of the above mentioned objectives are achieved by means of a system for identifying fluid condition inside a container, comprising: at least one fluid storage container, at least one resistive element associated with at least one fluid, at least one control unit of the resistive element associated with the resistive element.

According to the underlying premises of the invention in question, the system further comprises the fact that the resistive element is a resistance that varies with temperature.

Additionally, the system further comprises the fact that the resistive element includes a PTC-type resistor.

Moreover, the present invention proposes a system wherein the processing unit comprises an electronic control unit.

Further, according to the present invention, the system further comprises the fact that the control unit of the resistive element is a processing unit.

Additionally, in the present invention, the system comprises the fact that the control unit of the resistive element is a heater control unit associated with a processing unit.

In addition, the present invention describes a method for measuring the condition of fluids inside a container, which comprises the following steps: obtaining, by means of a control unit of the resistive element, the electrical reading of a resistive element inserted into a fluid storage container, processing the information obtained by the control unit of the resistive element by means of a processing unit, identifying, through the information processed by the processing unit, the conditions of the fluids in contact with the resistive element, and providing the information on the state of the fluids stored in the fluid storage container.

In the present invention, the method additionally identifies the state of the fluid by the processing unit associating with a lower limit and an upper limit of resistivity intrinsic to the material of the resistive element.

Moreover, the method of the present invention also comprises the fact that it has the step wherein the heater control unit increases the voltage applied to the resistive element in case the processing unit identifies freezing of the fluid contained inside the liquid storage container.

Finally, the present invention includes a method for measuring fluid condition inside a container which includes the fact that it is performed in a system for measuring the condition of a fluid inside a container, which has at least one fluid storage container, at least one resistive element associated with at least one fluid, and at least one heater control unit associated with the resistive element.

### BRIEF DESCRIPTION OF THE DRAWINGS

The preferred embodiment of the invention in question is described in detail on the basis of the drawings listed, wherein:

FIG. 1 illustrates, in perspective, the system for measuring a fluid condition inside a container.

FIG. 2 graphically illustrates the resistance and current as a function of time when the state of the fluid is of good quality.

FIG. 3 graphically illustrates the resistance and current as a function of time when fluid state is of intermediate quality.

FIG. 4 graphically illustrates the resistance and current as a function of time when fluid state is of poor quality.

FIG. 5 graphically illustrates the resistance and current as a function of time when the fluid storage container is empty of fluid.

FIG. 6 graphically illustrates the resistance and current as a function of time when the fluid is in the solid state.

### DETAILED DESCRIPTION

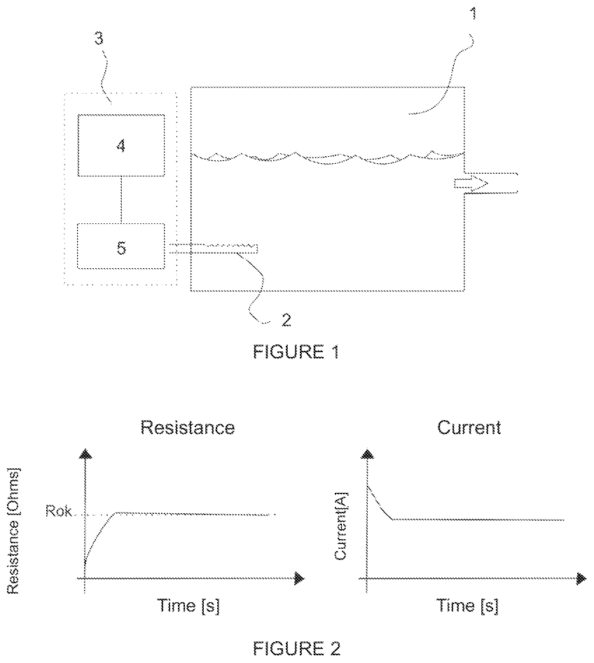

According to the general objectives of the invention in question, the system for identifying the condition of fluids inside a container comprises: at least one fluid storage container 1, at least one resistive element 2 associated with at least one fluid and at least one control unit of the resistive element 3 associated with the resistive element 2, as exemplified in FIG. 1.

In this way, the fluid storage container 1 may contain various types of fluids in the solid, liquid, and gaseous state. Internally associated with it, and consequently in contact with the internal fluids, the resistive element 2 comprises a resistance that varies with temperature, a PTC-type resistor or other temperature-sensitive elements, such as thermistors that work with a positive temperature coefficient.

Thus, the control unit of the resistive element 3 associated with the resistive element 2 is responsible for performing the reading of the resistive value, by applying a voltage to it, which varies depending on the fluid in which it is immersed.

The processing unit 4, which performs the processing of electrical signals obtained by the resistive element 2, comprises an electronic control unit (ECU). This signal processing can be any element provided with a processor and at least one memory unit that is able to read the electrical signals and transform them digitally, and by means of programming, to identify the fluid in which the resistive element 2 is immersed.

In this way, the control unit of the resistive element 3 comprises a processing unit 4, which, when associated with the resistive element 2, identifies the condition of the fluid element contained inside the fluid storage container 1.

Further, as an alternative embodiment, the control unit of the resistive element 3 also comprises a heater control unit 5 and a processing unit 4. The function of the heater control unit 5, associated with the resistive element 2 and the processing unit 4, is to provide a higher voltage to the resistive element 2 and consequently higher power when the condition of the fluid element is solid, and also to transmit the information on the reading of the resistive element 2 to the processing unit 4. In addition, if equipped with at least one processor and at least one memory, the heater control unit 5 can process and identify the state of the fluid in contact with the resistive element 2, transmitting the result to the processing unit 4. The association between the heater control unit 5 and the processing unit 4 can be carried out by physical connections by means of electrical signals, whether or not communication protocols are used, such as the CAN protocol.

This invention also discloses a method for measuring the condition of fluids inside a container, which comprises the steps of: obtaining, by means of a control unit of the resistive element 3, the electrical reading of a resistive element 2 inserted into a fluid storage container 1, processing the information obtained by the control unit of the resistive element 3 by means of a processing unit 4, identifying, through the information processed by the processing unit 4, the conditions of the fluids in contact with the resistive element 2, providing the information on state of the fluids stored in the fluid storage container 1.

Thus, the method described in this invention comprises the fact that the state of the fluid identified by the processing unit 4 is associated with a lower limit and an upper limit of resistivity associated with the material of the resistive element 2. Within this range of values, the fluid can be characterized in five states: good quality, intermediate quality, poor quality, empty container, and solid state fluid.

For the state in which the fluid is of good quality, by obtaining the voltage read in the resistive element 2, and consequently the resistance thereof, one can observe a value of an intermediate range in the resistive curve thereof, as shown in FIG. 2.

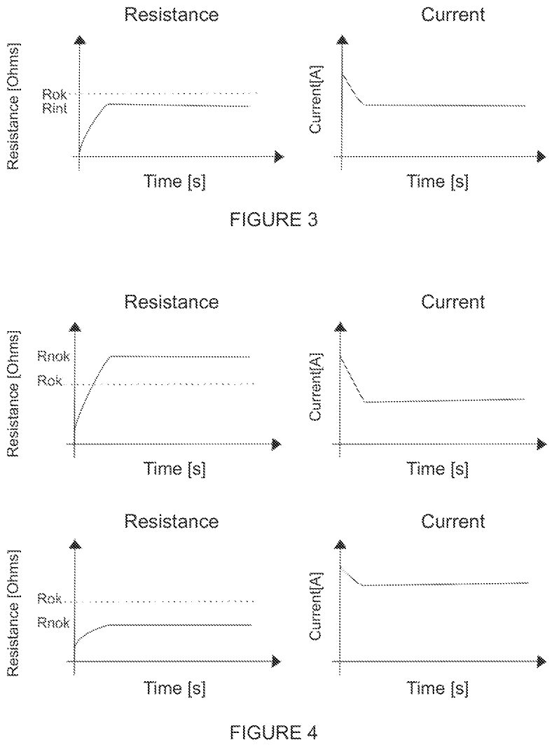

In a second state, in which the quality of the fluid is of an intermediate quality, an intermediate value of the resistance value of the resistive element 2 that is outside the value considered is considered for good quality, as shown in FIG. 3.

The third state of poor quality, the resistance value of the resistive element 2 is within two ranges closer to the upper and lower limit of the resistive curve of the resistive element, as shown in FIG. 4.

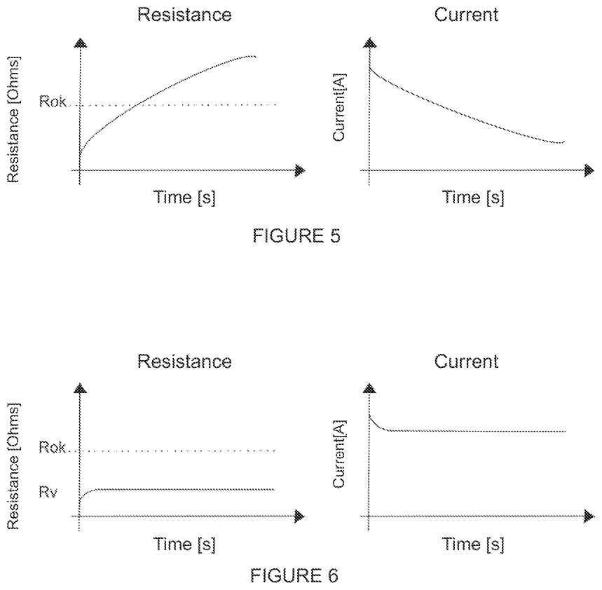

For the empty container, the resistance value of the resistive element 2 is at the upper limit of the resistive curve thereof, as shown in FIG. 5.

The last state observed in the proposed method is when the fluid is in the solid state, due to low temperatures. Accordingly, the resistance value of the resistive element 2 is at a lower threshold value of the resistance curve thereof, as shown in FIG. 6.

Additionally, the present invention describes a method that further comprises the step in which the heater control unit 3 increases the voltage applied to the resistive element 2 if the processing unit 4 identifies that the fluid contained inside the fluid storage container 1 is in the solid state.

Finally, it is worth mentioning that the method for measuring the condition of fluids inside a container comprises the fact that it is performed in a system for measuring the condition of fluids inside a container, composed of at least one fluid storage container 1, at least one resistive element 2 associated with at least one fluid, at least one heater control unit 3 associated with the resistive element 2.

It is important to highlight that the sole purpose of the above description is to describe one example of a particular embodiment of the invention in question. Therefore, it is clear that any modifications, variations, and constructive combinations of the elements that perform the same function substantially in the same way to achieve the same results, remain within the scope of protection delimited by the accompanying claims.

1. A system for identifying the condition of a fluid inside a container, the system comprising:
at least one fluid storage container (1);
the system being CHARACTERIZED by further comprising:
at least one resistive element (2) associated with at least a fluid;
at least one control unit of the resistive element (3) associated with the resistive element (2).

2. The system according to claim 1, CHARACTERIZED in that the resistive element (2) comprises a resistance that varies with the temperature.

3. The system according to claim 1, CHARACTERIZED in that the resistive element (2) comprises a PTC-type resistor.

4. The system according to claim 1, CHARACTERIZED in that the control unit of the resistive element (3) comprises a processing unit (4).

5. The system according to claim 4, CHARACTERIZED in that the processing unit (4) comprises an electronic control unit.

6. The system according to claim 1, CHARACTERIZED in that the control unit of the resistive element (3) comprises a heater control unit (5) and a processing unit (4).

7. A method for measuring the condition of fluids inside a container, CHARACTERIZED by comprising the steps of:
obtaining, with a control unit of the resistive element (3), the electric reading of a resistive element (2) inserted into a fluid storage container (1);
processing the information obtained by the control unit of the resistive element (3) with a processing unit (4);
identifying, through the processed information by the processing unit (4), the conditions of the fluids in contact with the resistive element (2); and
providing the information on the state of the fluids stored in the fluid storage container (1).

8. The method for measuring the condition of fluids inside a container according to claim 7, CHARACTERIZED in that the state of the fluid identified by the processing unit (4) is associated with a lower limit and an upper limit of resistivity associated with the material of the resistive element (2).

9. The method for measuring the condition of fluids inside a container according to claim 7, CHARACTERIZED in that the method further comprises the step of the control unit of the heater (3) increasing the voltage applied to the resistive element (2) if the processing unit (4) identifies that the fluid contained inside the fluid storage container (1) is in solid state.

10. The method for measuring the condition of fluids inside a container according to claim 7, CHARACTERIZED by being executed in a system for reading the condition of fluids inside a container, comprising:
at least one fluid storage container (1);
at least one resistive element (2) associated with at least one fluid; and
at least one control unit of the heater (3) associated with the resistive element (2).
